(19) United States
(12) Patent Application Publication
(10) Pub. No.: US 2006/0223113 A1
(43) Pub. Date: Oct. 5, 2006

(54) Immobilization of binding agents

(75) Inventors: Hans Sjobom, Uppsala (SE); Stefan Lofas, Uppsala (SE)

Correspondence Address: SEED INTELLECTUAL PROPERTY LAW GROUP PLLC, 701 FIFTH AVE, SUITE 6300, SEATTLE, WA, 98104-7092, US

(73) Assignee: Biacore AB, Uppsala (SE)

(21) Appl. No.: 11/266,022

(22) Filed: Nov. 3, 2005

Related U.S. Application Data
(63) Continuation of application No. 10/327,708, filed on Dec. 20, 2002, now abandoned.
(60) Provisional application No. 60/343,343, filed on Dec. 21, 2001.

(30) Foreign Application Priority Data
Dec. 21, 2001 (SE) 0104365-2

(51) Int. Cl. G01N 33/53; C12M 1/34

(52) U.S. Cl. 435/7.1; 435/287.2; 427/2.11

(57) ABSTRACT

A method of preparing an array of discrete localized binding agent-supporting regions on a solid support surface as well as an array prepared by the method are disclosed. The method comprises: providing a solid support bearing functional groups at a plurality of predefined discrete regions and being in a first surface property state; activating the functional groups to reactive groups to convert any activated surface area to a second surface property state which differs substantially from that of the first state; selectively dispensing at each predefined discrete region a predetermined volume of a liquid medium containing a binding agent to couple the binding agent to the reactive groups; and deactivating unreacted reactive groups such that the first surface property state of the solid support surface is restored. The use of the method for studying molecular interactions and for performing assays for one or more analytes are also disclosed.

### CROSS-REFERENCES TO RELATED APPLICATIONS

This application is a continuation of U.S. patent application Ser. No. 10/327,708 filed Dec. 20, 2002, now pending; which claims the benefit under 35 U.S.C. §119(e) of U.S. Provisional Patent Application No. 60/343,343 filed Dec. 21, 2001, and also claims priority to Swedish Application No. 0104365-2 filed Dec. 21, 2001; all of these applications are incorporated herein by reference in their entireties.

### BACKGROUND OF THE INVENTION

1. Field of the Invention

The present invention relates to spatially-addressable immobilization of binding agents on a support surface, and more particularly to a method of preparing an array by spatially immobilizing binding agents at predefined regions on a support surface, an array prepared by the method, the use of the prepared array, a biosensor comprising the array, and an activated solid support surface to which binding agents can be coupled.

2. Description of the Prior Art

Array technology providing arrays of binding agents, for example ligands, such as oligonucleotides and peptides, on solid supports has become increasingly important in especially the biotechnological and pharmaceutical fields as a tool for performing repetitive assays and screenings of analytes, including gene expression analysis, drug screening, nucleic acid sequencing, mutation analysis, and the like.

Among the techniques used so far for dispensing, or “spotting”, the binding agents at discrete positions on the array may be mentioned piezoelectric micropipetting (“ink-jet”), micro-contact printing, capillary stamping, electrical addressing, and inertia-driven ejection of microdrops. For general reviews of so-called microarray technology it may be referred to, for example, Tibtech 1996, 14: 401-407 and Tibtech 1996, 16: 301-306.

An important step in the preparation of microarrays is the immobilization of the binding agents on the support surface. A large variety of methods are known for attaching biomolecules to solid supports, including covalent bonding to the support surface and non-covalent interaction of the biomolecules with the surface.

General descriptions of binding methods are given in, for example, Affinity Techniques. Enzyme Purification: Part B. Methods in Enzymology, Vol. 34, ed. W. B. Jacoby, M. Wilchek, Acad. Press, N.Y. (1974), and Immobilized Biochemicals and Affinity Chromatography, Advances in Experimental Medicine and Biology, vol. 42, ed. R. Dunlop, Plenum Press, N.Y. (1974). Exemplary binding methods are also disclosed in the following publications.

U.S. Pat. No. 4,681,870 describes a method for introducing free amino or carboxyl groups onto a silica matrix. These groups may subsequently be covalently linked to, e.g., a protein or other ligand, in the presence of a carbodiimide. Alternatively, a silica matrix may be activated by treatment with cyanogen halide under alkaline conditions. The ligand is covalently attached to the surface upon addition to the activated surface.

U.S. Pat. No. 4,282,287 describes a method for modifying a polymer surface through successive application of multiple layers of biotin, avidin and extenders.

U.S. Pat. No. 4,762,881 describes a method for attaching a polypeptide to a solid substrate by incorporating a light-sensitive unnatural amino acid group into the polypeptide chain and exposing the product to low-energy ultraviolet light.

Modification of surface characteristics in terms of hydrophilic and hydrophobic properties in the preparation of microarrays has also been described in the prior art.

For example, WO 98/42730 discloses the preparation of a support surface which is hydrophilic in a first state and hydrophobic in a second state, so that the substrate can be used in either aqueous or organic media. The substrate surface contains a plurality of hydrophilic sites which can be readily protected and deprotected. By protecting a fraction of the sites, the surface is provided in a hydrophobic form such that the unprotected sites may participate in organic synthesis processes to be conducted using organic reagents and solvents. Following synthesis, the protected hydrophilic sites are deprotected, regenerating the substrate surface in hydrophilic form for use with aqueous reagents, e.g., in screening and/or separation procedures to be conducted in aqueous media.

U.S. Pat. No. 6,127,129 discloses a method of making a biomolecule or cellular array on a metal substrate. An ω-modified alkanethiol monolayer is deposited on the metal substrate to form a hydrophilic surface. The monolayer is then reacted with hydrophobic protecting groups, and the surface is photopatterned to create an array of exposed metal surface areas. Subsequent deposition of ω-modified alkanethiol in the areas of exposed metal substrate yields an array of discrete hydrophilic spots of unprotected ω-modified alkanethiol, to which biomolecules or cells are attached. The protecting groups are then removed from the hydrophobic background surrounding the discrete spots with immobilized biomolecules or cells, and the monolayer is made resistant to non-specific protein binding, e.g., by attaching PEG moieties thereto.

EP-A-895 082 discloses a method for spotting probes on a solid support which comprises reacting maleimido groups on the support surface with thiol group-containing nucleic acid probes added by an ink-jet method. An alternative binding pair is epoxy groups on the surface and amine groups on the nucleic acid probes. Non-specific binding to the surface is prevented by blocking with BSA solution or decomposition of unreacted maleimido groups. A surface containing epoxy groups may be made hydrophilic by opening unreacted epoxy rings with ethanolamine to form hydroxy groups after binding of the nucleic acid probes.

WO 98/55593 discloses the patterning of a solid support with synthetic nucleic acid molecules using, for example, an ink-jet printing delivery technique. The support surface is silanized providing a very hydrophobic surface which allows oligonucleotide probe droplets to form at specific and localized positions on the solid support surface with no cross contamination between probes even at high probe density.

WO 98/39481 discloses methods for covalent, specific immobilization of nucleic acid molecules onto a solid mercaptosilanized hydrophobic surface by a reversible disulfide bond formed by coupling a sulfhydryl- or disulfide-modified nucleic acid molecule to the sulfhydryl groups of the mercaptosilanes. A high probe density without cross contamination may be achieved.

U.S. Pat. No. 6,066,448 discloses methods for producing patterned multi-array, multi-specific surfaces. The binding domains on the multi-array surface may be hydrophobic or hydrophilic, and the surrounding surface may have the opposite property (hydrophilic or hydrophobic) to that of the binding domains. The use of such a hydrophilic/hydrophobic border aids in confining the produced binding domain to a discrete area on the surface. The hydrophobic and hydrophilic binding domains may be generated by micro-contact printing.

U.S. Pat. No. 5,474,796 discloses the preparation of array plates by coating a glass plate with a photoresist substance, exposing to light to obtain a patterned surface of exposed and photoresist-coated surfaces, reacting the exposed surfaces with a hydrophobic reagent, removing the photoresist, and converting the exposed surface areas to hydrophilic binding regions. Alternatively, one starts from a derivatized hydrophilic surface to directly obtain the hydrophilic binding regions in the last step.

U.S. Pat. No. 5,985,551 discloses the preparation of an array plate which comprises a support surface having a covalently linked layer of inert siloxane defining an array of 10 to 104 sites per cm² which do not have the covalently linked layer. Chemical reactant solutions are localized to these sites, which are about 50-2000 microns in diameter, via surface tension.

U.S. Pat. No. 6,171,797 discloses a method for making arrays of distinct polymers covalently bonded to the surface of a solid support. At least two distinct polymers, e.g., nucleic acids, are covalently bound to the support surface through a cycloaddition reactive group on the surface capable of reacting with a group present on the polymers in a cycloaddition reaction to produce a covalent linkage between the polymer and the support surface. In many embodiments, the contact angle of the cycloaddition reactive group is sufficient to provide for extremely low drop spreading of fluid deposited on the substrate surface.

WO 01/94946 discloses the preparation of arrays of protein-binding agents. In one embodiment, a gold-surfaced microscope slide is coated with an aminothiol layer that is then functionalized with a group that will bind to an anchor functional group, e.g., a thiol function, of a protein binding agent. After deposition of the protein binding agent(s) to the generally hydrophilic functionalized surface, unreacted maleimides on the surface can be blocked chemically, e.g., with a hydrophilic thiol, to minimize background non-specific binding of proteins. The surface can also be blocked using proteins, such as human serum albumin (HSA).

U.S. Pat. No. 5,624,711 discloses the preparation of supports for solid phase synthesis of oligomer arrays of single compounds. A glass slide or other support is derivatized with an aminoalkylsilane to provide a surface of amine functional groups. This surface is then treated with a mixture of linking molecules (e.g., nitroveratryloxycarbonyl-aminocaproic acid) and diluent molecules (e.g., protected amino acids) to provide a surface having initiation sites at a preselected density. The linking molecule contributes to the net hydrophobic or hydrophilic nature of the surface and can be selected to improve presentation of the polymer synthesized thereon to certain receptors, proteins or drugs. The diluent molecules can also be selected to impart hydrophobic or hydrophilic properties to the substrate surface. For example, o-t-butylserine as a diluent molecule provides a hydrophobic surface during polymer synthesis but upon treatment with acid, ether cleavage provides a more hydrophilic surface for assays.

U.S. Pat. No. 6,329,209 discloses arrays of different protein capture agents immobilized on discrete patches, e.g., of gold, covered by an organic thinfilm. The surfaces, or border regions, between the patches of protein-capture agents may be covered by a different organic thinfilm with low non-specific binding properties for proteins and other analytes, e.g., a monolayer of hydrophilic chains attached to an ordered hydrophobic monolayer of alkyl chains.

Generally, however, the prior art methods for attaching binding agents to surfaces are unsatisfactory in several respects. Deficiencies include low reaction efficiencies and a general inability to readily permit regional and selective attachment of a plurality of binding agents to the surface. Also, high non-specific binding of analytes to the surface is often observed when performing screenings and assays using ligand-supporting surfaces prepared according to the prior art methods. There is therefore a need of a more efficient and simpler to perform method of binding agent immobilization in the preparation of arrays. Such a method should provide stable attachment of selected binding agents to predefined surface regions, yet the attachment should be strictly restricted to the predefined regions. Also, when performing assays and screenings using the prepared surfaces, non-specific binding of analytes to the surfaces should be low or negligible. The above needs are fulfilled by the present invention, which provides further related advantages.

### BRIEF SUMMARY OF THE INVENTION

In brief, the present invention relates to the preparation of arrays of binding agents and is based on the concept of utilizing activation of functional groups to reactive (or more reactive) groups to thereby temporarily change the hydrophilic/hydrophobic properties of a functional group-bearing solid support surface. After contacting predefined regions or spots on the surface with liquid medium containing binding agent to form the array of immobilized binding agents, deactivation of the surface brings the surface back to its original hydrophilic or hydrophobic state. Such a temporary change of the surface properties may be used in different ways to improve the formation of a desired array.

For example, temporarily making a generally hydrophilic support surface area substantially less hydrophilic, or even hydrophobic, by activating functional groups on the hydrophilic surface area (at least at predefined regions or spots thereof) to reactive groups which are sparingly hydrophilic or, preferably, hydrophobic, and then contacting the surface area at the predefined regions or spots with one or more aqueous liquids containing binding agent to be immobilized on the surface, may reduce spreading or extension of liquid on the surface and the immobilization of binding agent may be effectively restricted to the predefined regions. After conversion or deactivation of any unreacted reactive groups to more hydrophilic groups, the original generally hydrophilic character of the surface is restored and non-specific binding to the surface is minimized when subsequently using the surface for analysis.

Likewise, hydrophobic functional groups on a generally hydrophobic surface area may be activated to hydrophilic groups to permit coupling of binding agents at defined regions or spots on the surface, and unreacted reactive groups are then converted or deactivated to hydrophobic groups.

Therefore, in one aspect, the present invention provides a method of preparing an array of discrete localized binding agent-supporting regions on a solid support surface, which method comprises the steps of:

providing a solid support having a surface bearing functional groups at least at a plurality of predefined discrete regions thereon, which surface is in a first surface property state,

reacting functional groups on at least the predefined discrete regions of the solid support surface with an activating agent selected to activate the functional groups to reactive groups of such a polarity that any activated surface area is provided in a second surface property state which differs substantially from that of the first state,

selectively dispensing at each predefined discrete region on the solid support surface a predetermined volume of a liquid medium containing a binding agent to couple the binding agent to the reactive groups, and

deactivating unreacted reactive groups on the solid support surface such that the first surface property state of the solid support surface is restored.

In one embodiment, substantially the whole functional group-bearing solid phase surface is subjected to activation conditions. In another embodiment, the activation of functional groups is restricted to the predefined discrete regions on the support surface.

In a preferred embodiment of the invention, there is provided a method of preparing an array of discrete localized binding agent-supporting regions on a solid support surface, which method comprises the steps of:

providing a solid support having a surface bearing functional groups such that the surface in a first state is hydrophilic,

defining a plurality of predefined discrete regions on the solid support surface,

reacting functional groups on at least the predefined discrete regions of the solid support surface with an activating agent selected to activate the functional groups to less polar reactive groups such that any activated surface area is provided in a second state which is at least substantially less hydrophilic than the first state,

selectively dispensing at each predefined discrete region on the solid support surface a predetermined volume of an aqueous liquid containing a binding agent to couple the binding agent to the reactive groups, the second less hydrophilic state of the activated surface regions substantially preventing extension and spreading of binding agent-containing aqueous liquid when applied thereto, and

deactivating unreacted reactive groups on the solid support surface such that the first hydrophilic state of the solid support surface is restored.

In another aspect, the present invention provides an array of one or more binding agents prepared by the method according to the first aspect.

Other aspects of the present invention provide the use of an array prepared by the method according to the first aspect for studying molecular interactions, and for performing an assay for one or more analytes, respectively.

Still another aspect of the invention relates to a biosensor comprising an array prepared by the method according to the first aspect.

Yet another aspect of the invention relates to an activated solid support surface to which binding agents can be coupled.

### DEFINITIONS

In describing and claiming the present invention, the following terminology will be used in accordance with the definitions set out below.

“Array” as used herein generally relates to a linear or two-dimensional array of discrete regions, each having a finite area, formed on a continuous surface of a solid support and supporting one or more binding agents. Ordered arrays of nucleic acids, proteins, small molecules, cells or other substances on a solid support enable parallel analysis of complex biochemical samples. In a “microarray”, the density of discrete regions, or spots, is typically at least 100/cm², and the discrete regions typically have a diameter in the range of about 10-1000 μm, usually about 10-500 μm and are separated from other regions in the array by about the same distance.

“Predefined region” as used herein relates to a localized area on the solid support surface. The predefined region may have any desired shape, such as circular, rectangular, elliptical, etc, and is below often referred to as a “spot”.

37 Solid support” as used herein is meant to comprise any solid (flexible or rigid) substrate onto which it is desired to apply an array of one or more binding agents. The substrate may be biological, non-biological, organic, inorganic or a combination thereof, and may be in the form of particles, strands, precipitates, gels, sheets, tubings, spheres, containers, capillaries, pads, slices, films, plates, slides, etc., having any convenient shape, including disc, sphere, circle, etc. The substrate surface supporting the array may have any two-dimensional configuration and may include, for example steps, ridges, kinks, terraces and the like and may be the surface of a layer of material different from that of the rest of the substrate.

“Functional group” as used herein means a reactive chemical entity that serves to connect a binding agent to the surface. Usually, functional groups need to be activated in order to immobilize a binding agent. The functional groups may be inherently present in the material used for the solid support or they may be provided by treating or coating the support with a suitable material. The functional group may also be introduced by reacting the solid support surface with an appropriate chemical agent.

“Activation” as used herein means a modification of a functional group on the solid support surface to enable coupling of a binding agent to the surface.

“Binding agent” as used herein means any agent that is a member of a specific binding pair, including, for instance polypeptides, such as proteins or fragments thereof; nucleic acids, e.g., oligonucleotides, polynucleotides, and the like; etc. The binding agent is often a ligand.

“Ligand” as used herein means a molecule that has a known or unknown affinity for a given analyte and can be immobilized on a predefined region of the surface. The ligand may be a naturally occurring molecule or one that has been synthesized. The ligand may be used per se or as aggregates with another species. Optionally, the ligand may also be a cell.

“Analyte” as used herein is a molecule, typically a macromolecule, such as a polynucleotide or polypeptide, the presence, amount and/or identity of which are to be determined. The analyte is recognized by a particular ligand forming an analyte/ligand pair. Optionally, the ligand may also be a cell.

“Surface property” as used herein relates to the hydrophilic or hydrophobic character of a surface.

“Hydrophobic” as used herein may be defined as water-repelling whereas “hydrophilic” may be defined as water-attracting. With regard to a surface, the terms hydrophobic and hydrophilic may be defined by the contact angle for a droplet of a liquid on a planar solid surface, the contact angle being measured from the plane of the surface, tangent to the surface at the three phase boundary line. A hydrophilic liquid will thus have a low contact angle on a hydrophilic surface, whereas a hydrophobic liquid will have a high contact angle. For example, hydrophobic surfaces typically have contact angles with water in the range of 40 to 110°, while the contact angles with water for hydrophilic surfaces typically are in the range of 1 to 25°. Thus, a support surface is hydrophobic if an aqueous medium droplet applied to the surface does not spread out substantially beyond the area size of the applied droplet, the surface acting to prevent spreading of the droplet applied to the surface by hydrophobic interaction with the droplet.

“Differ substantially” as used herein with regard to surface property states means that a surface in one state is substantially less hydrophilic or substantially less hydrophobic than in the other state.

“Substantially less hydrophilic” as used herein usually means a reduction in hydrophilicity by an increase in contact angle of at least about 10 degrees, preferably at least about 15 degrees. When it is said, for example, that a surface is made substantially less hydrophilic, this includes, of course, also that a hydrophilic surface may be made hydrophobic. Likewise, when it is said that a surface is made substantially less hydrophobic, this includes, of course, also that a hydrophobic surface may be made hydrophilic.

“Polarity” as used herein refers to the polar/non-polar properties of a chemical group. “Polar” and “non-polar” are related to the terms hydrophilic and hydrophobic. A polar group is hydrophilic, and a non-polar group is hydrophobic. A hydroxy group is an example of a polar group, and an alkyl group is an example of a non-polar group. When it is said, for example, that a group is “less polar”, this includes, of course, also that the group may be non-polar, and vice versa.

The term “aqueous liquid medium” as used herein refers to a liquid medium containing less than about 50 vol. % of organic solvent, more preferably less than about 10 vol. % organic solvent, and most preferably about 0 vol. % organic solvent.

The terms “non-aqueous liquid medium” and “organic liquid medium” as used herein refer to a liquid medium containing less than about 50 vol. % of water, more preferably less than about 10 vol. % water, and most preferably about 0 vol. % water.

In the specification and the appended claims, the singular forms “a”, “an”, and “the” are meant to include plural reference unless it is stated otherwise. Also, unless defined otherwise, technical and scientific terms used herein have the same meaning as commonly understood to a person skilled in the art related to the invention.

### DETAILED DESCRIPTION OF THE INVENTION

As mentioned above, the present invention relates to the coupling of binding agents, such as ligands, to functional groups on an solid support surface at predefined regions, or spots, thereon to prepare an array of the binding agents on the surface. Usually, it is desired that the array surface exhibit a generally hydrophilic character to prevent nonspecific binding of biomolecular analytes thereto when using the array surface in assays. On the other hand, it is also desired that at least the predefined regions on the array surface have a substantially less hydrophilic character than the rest of the array surface, and preferably a generally hydrophobic character, to prevent extensive spreading of droplets of aqueous liquid containing the binding agents when contacting the surface therewith to couple the binding agents to the surface.

The invention favourably makes use of activation of the functional groups to reactive groups with selected activating agents to provide a substantially less hydrophilic, or preferably hydrophobic surface to be contacted with the binding agent-containing liquid droplets. In this way, a hydrophilic surface may temporarily be made much less hydrophilic or even hydrophobic and thereby satisfy both the above needs, i.e., exhibit an at least almost hydrophobic character for the coupling of the binding agents to substantially reduce or eliminate the spreading of the binding agents on the surface, and after the coupling provide the desired hydrophilic character of the non-coupled areas to reduce or eliminate non-specific binding. Due to the low spreading on the activated surface areas, the binding agent-supporting regions may be confined and well defined and, if desired, permit a high density of binding agent-regions or spots, despite the use of a water-based binding agent-containing liquid.

In case the medium containing the binding agent is an organic liquid medium, the inventive concept may be used in the opposite way, i.e., to temporarily create a hydrophilic surface, or hydrophilic spots on a hydrophobic surface, to concentrate a deposited non-aqueous (organic) droplet on the surface and thereby reduce or eliminate the spreading of the binding agents on the surface. Such a temporary creation of a hydrophilic surface or hydrophilic spots on a hydrophobic surface may, however, also be used when depositing droplets of an aqueous liquid medium containing binding agent. As an example hereof may be mentioned the immobilization of proteins to a surface supporting a hydrophobic lipid mono- or bilayer.

In one method embodiment of the invention, functional groups on the surface are converted to reactive groups by selective activation of the functional group on predefined spots on the surface. The activated reactive groups are then used to immobilize binding agents, e.g., ligands, at the predefined spots by dispensing a predetermined liquid volume containing binding agent at each spot. The procedure may be repeated at different sites on the surface so as to provide a surface prepared with a plurality of spots on the surface which contain the same or different binding agents. If the binding agents are ligands having a particular affinity for one or more analytes, screenings and assays can be conducted in the regions of the surface containing the ligands, as will be further described below. In some embodiments, a first binding agent bound to the solid support surface serves as a capturer for a second binding agent, which may be a ligand. After preparation of the array, the surface may be dried and stored for subsequent use, if desired.

In an alternative method embodiment, functional groups on the entire surface are activated rather than only at the predefined spots, and the binding agents are then selectively bound to the predefined spots.

The selective dispensing of the predetermined liquid volumes to the predefined spots may preferably be performed by a deposit device as will be described further below.

The use of activation according to the present invention thus enables (preferably covalent) coupling of the binding agents and at the same time provides the desired temporary surface property to the immobilization regions or spots, such as e.g., making a hydrophilic surface substantially less hydrophilic in order to prevent aqueous solutions of binding agents from spreading when contacted with the activated regions. This in turn aids in concentrating and confining the immobilization of binding agents to the predefined regions. An exemplary schematic illustration of the method of the invention is given below.

The activation (A) of functional groups (F) on a surface (S) of a solid substrate may generally be illustrated by the following equation:
S-F+A→S-F-A

Predefined regions, or spots, (Sᵢ) on the surface may be activated for ultimate immobilization of ligands at the predefined regions by selectively dispensing (depositing) an activation mixture at the predefined regions to convert functional groups to activated groups. The process is illustrated by the equation:
Sᵢ-F+A→Sᵢ-F-A

Immobilization of a ligand (Lᵢ) on a predefined region of the surface is illustrated by the equation:
Sᵢ-F-A+Lᵢ→Sᵢ-F-Lᵢ

Immobilization of a different ligand (Lⱼ) on a different region (Sⱼ) of the surface may be shown by the equation:
Sⱼ-F-A+Lⱼ→Sⱼ-F-Lⱼ

Repetition of the above steps on different regions of the surface will produce a matrix of ligands immobilized on the substrate surface. Such a matrix may have any desired pattern of ligands.

An immobilized ligand on a surface will have a specific binding affinity for a particular analyte (An). An example of a direct assay at a predefined region of the surface for the presence of the analyte (An′) in a liquid medium is illustrated by the equation:
Sᵢ-F-Lᵢ+An′→Sᵢ-F-Lᵢ-An′

The original hydrophilic or hydrophobic character of the substrate surface bearing the functional group may be provided by (i) the functional group only, (ii) both the functional group and the substrate surface material, or (iii) the substrate surface material only. In the first-mentioned case (i), for a hydrophilic surface for example, the functional group should be relatively hydrophilic, whereas in case (ii) the functional group may also have a moderate or low hydrophilicity. In case (iii), the functional group may even be slightly hydrophobic as long as the substrate surface material otherwise has a sufficient hydrophilicity to make the surface generally hydrophilic.

A wide variety of activatable functional groups well-known to the skilled person may be used in the invention. Examples of such hydrophilic groups are hydroxy, carboxy, carbonyl, formyl, amino, and mercapto groups, just to mention a few.

Methods for activating the functional groups are readily apparent to the skilled person and may be selected from a wide variety of methods. In the case of a hydrophilic starting surface, for example, the activating agent is selected to provide an activated surface, or activated surface regions, having a substantially reduced hydrophilicity, and preferably one that is hydrophobic. Such an activated surface or activated surface regions should preferably have a contact angle (as defined above) in the range of from about 20 to about 100°, particularly from about 40 to about 100°, and especially from about 60 to 100°.

Activating agents and methods that may be used depend, of course, on the functional group to be activated and on the desired reactive group to be obtained by the activation. Exemplary functional group/activating agent combinations for a hydrophilic surface include those introducing hydroxysuccinimide esters, nitro- and dinitrophenyl esters, tosylates, mesylates, triflates and disulfides. For example, a hydroxy group may be reacted to activated ester with disuccinic carbonate, or to epoxide with a diepoxide. A carboxy group may be activated to N-hydroxysuccinimide ester by reaction with N-hydroxysuccinimide and carbodiimide, or to dinitrophenyl ester by reaction with dinitrophenol. A thiol (mercapto) group may be activated to a disulfide group by reaction with dipyridyldisulfides.

To immobilize the binding agents on the surface, the activated functional groups, which, as mentioned above, may be present on the predefined regions only, or alternatively on the entire surface, are reacted selectively at the predefined regions with the binding agent or agents. The necessary reaction conditions, including time, temperature, pH, solvent(s), additives, etc will depend on inter alia the particular species used and appropriate conditions for each particular situation will readily be apparent to the skilled person.

The liquid medium containing the binding agent, usually a solution thereof, may be applied to the respective predefined region by selectively depositing a predetermined volume of the liquid on the surface of the solid support. Such deposition may be performed manually, e.g., via a pipette, or through the use of an automated machine or device. A wide variety of devices for dispensing or depositing aqueous solutions onto precise locations of a support surface, so-called “arrayers”, are known to the skilled person and may be employed in the present invention. Such devices include “ink-jet” printing devices (including piezoelectric and thermal devices), “pin-and-ring” devices, devices for micro-contact printing, devices for capillary stamping, and the like. Reference may be made to, for example, U.S. Pat. No. 4,877,745, U.S. Pat. No. 5,338,688, U.S. Pat. No. 5,474,996, U.S. Pat. No. 5,658,802, U.S. Pat. No. 6,165,417, WO 00/56455, WO 00/56433, WO 00/56442, and WO 98/51999, the disclosures of which are incorporated herein by reference. Commercial arrayers are available from a number of vendors, including e.g., Packard, Biorobotics, Affymetrix, Techan, Cartesian Technologies, Gene Machines, Molecular Dynamics, and HSG-IMIT.

The predetermined liquid volume to be dispensed onto each individual surface region or spot depends on inter alia the spot size, the density of the functional group, the binding agent and its concentration, the deposit device, etc, and suitable liquid volumes in each particular situation may readily be selected by the skilled person.

When, for example, a hydrophilic surface is temporarily made less hydrophilic, the less hydrophilic or even hydrophobic character of the surface regions supporting the activated functional groups will provide for extremely low spreading of an aqueous fluid deposited on the surface. The resulting spots of immobilized binding agent on the surface may therefore be rather small and usually have a diameter below about 1 mm, particularly below about 500 μm, and especially do not exceed about 200 μm. On the other hand, the spot diameter is preferably not smaller than about 1 μm, more preferably not smaller than about 5 μm, and especially not smaller than about 10 μm. The above is, of course, also true when depositing a non-aqueous fluid on a hydrophobic surface that has temporarily been made hydrophilic.

Depending on the intended application of the array to be prepared, the same or different binding agents, or groups of different binding agents, may be bound to the plurality of predefined regions on the solid support surface.

After immobilizing the binding agent or agents to the surface, any residual reactive groups may be inactivated by treatment with an appropriate wash solution to restore the generally hydrophilic character of the surface. Such inactivating agents are well known to the skilled person and may readily be selected depending on the particular reactive groups on the surface.

The solid support is preferably a rigid structure and may comprise a substrate having a surface layer of a different material. Exemplary substrate materials are Langmuir Blodgett films, functionalized glass, Si, Ge, GaAs, GaP, SiO₂, SiN₄, modified silicon, and a wide variety of polymers, such as (poly)tetrafluoroethylene, (poly)vinylidenedifluoride, or combinations thereof. A preferred substrate material for many applications is flat glass.

The surface of the solid support may be composed of a variety of materials, for example, polymers, plastics, resins, polysaccharides, silica or silica-based materials, carbon, metals, inorganic glasses, membranes, etc, provided that the surface may support functional groups. A suitable surface is a metal film, e.g., gold, silver, or aluminum, preferably gold.

The solid support surface may be provided with a layer of a polymer. In such a case the polymers will carry the functional groups to be activated. The polymer may be selected from any suitable class of compounds, for example, polyethylene glycols, polyethylene imides, polysaccharides, polypeptides, or polynucleotides, just to mention a few. Attachment of the polymers to the support surface may be effected by a variety of methods that are readily apparent to a person skilled in the art. For example, polymers bearing trichlorosilyl or trisalkoxy groups may be reacted with hydroxyl groups on the substrate surface to form siloxane bonds. Attachment to a gold or silver surface may take place via thiol groups on the polymer. Alternatively, the polymer may be attached via an intermediate species, such as a self-assembled monolayer of alkanethiols. The type of polymers selected, and the method selected for attaching the polymers to the surface, will thus depend on the polymer having suitable reactivity for being attached to the substrate surface, and on the properties of the polymers regarding non-specific adsorption to, especially, proteins.

The functional groups may be present on the polymer or may be added to the polymer by the addition of single or multiple functional groups. Such functional groups are preferably heterobifunctional, having one end adapted to react with reactive groups on the polymer and the other end adapted to react with the activating agent. Methods for attaching the functional groups are readily apparent to the person skilled in the art and may be selected from a wide variety of groups. Exemplary methods are the amidation of amine groups on the polymer with succinic acid, and the reaction of hydroxy groups on the polymer with bromoacetic acid.

As mentioned above, the binding agent is usually a ligand, i.e., a molecule capable of recognizing a particular analyte in solution. Examples of ligands include, without any limitation thereto, agonists and antagonists for cell membranes, toxins and venoms, viral epitopes, antigenic determinants, hormones and hormone receptors, steroids, peptides, enzymes, substrates, cofactors, drugs, lectins, sugars, oligonucleotides, oligosaccharides, proteins, glycoproteins, cells, cellular membranes, organelles, cellular receptors, vitamins, and immunoglobulins, e.g., monoclonal and polyclonal antibodies.

Among ligands of particular interest may be mentioned those mediating a biological function on binding with a particular analyte(s). Suitable ligands are often relatively small single molecules, such as cofactors, which exhibit specific binding properties. Typically, ligands will have a size larger than about 100 D, especially larger than about 1 kD. Other examples of (currently) interesting ligands include, without any restriction thereto, the common class of receptors associated with the surface membrane of cells and include, for instance, the immunologically important receptors of B-cells, T-cells, macrophages and the like.

Analytes that may be assayed for include, without any restriction thereto, agonists and antagonists for cell membrane receptors, toxins and venoms, viral epitopes, hormones (e.g., opiates, steroids, etc), hormone receptors, peptides, enzymes, enzyme substrates, cofactors, drugs, lectins, sugars, oligonucleotides, oligosaccharides, proteins, and monoclonal antibodies.

An array surface prepared according to the method of the invention described above may be used for screening analytes having affinity for the immobilized ligands. Such a screening assay may be performed by contacting a solution containing an analyte (or analytes) with the surface for a suitable period of time. By determining those regions on the surface to which the analyte (or analytes) associates when the analyte is contacted with the surface, the ligands having affinity for the analyte may be identified.

Methods for detecting the presence of bound analyte(s) on the surface may be chosen from a wide variety of detection techniques, including, for example, marker-based techniques, where the analyte(s) or an analyte specific reagent is labelled, e.g., with a radiolabel, a chromophore, fluorophore, chemiluminescent moiety or a transition metal.

For many applications, the assays are performed with a biosensor. Biosensors may be based on a variety of detection methods. Typically such methods include, but are not limited to, mass detection methods, such as piezoelectric, optical, thermo-optical and surface acoustic wave (SAW) device methods, and electrochemical methods, such as potentiometric, conductometric, amperometric and capacitance methods. With regard to optical detection methods, representative methods include those that detect mass surface concentration, such as reflection-optical methods, including both internal and external reflection methods, angle, wavelength or phase resolved, for example ellipsometry and evanescent wave spectroscopy (EWS), the latter including surface plasmon resonance (SPR) spectroscopy, Brewster angle refractometry, critical angle refractometry, frustrated total reflection (FTR), evanescent wave ellipsometry, scattered total internal reflection (STIR), optical wave guide sensors, evanescent wave-based imaging such as critical angle resolved imaging, Brewster angle resolved imaging, SPR angle resolved imaging, and the like. Further, photometric methods based on, for example, evanescent fluorescence (TIRF) and phosphorescence may also be employed, as well as waveguide interferometers.

One exemplary type of SPR-based biosensors is sold by Biacore AB (Uppsala, Sweden) under the trade name BIACORE® (hereinafter referred to as “the BIACORE instrument”). These biosensors utilize a SPR based mass-sensing technique to provide a “real-time” binding interaction analysis between a surface bound ligand and an analyte of interest. A typical output from the BIACORE instrument is a “sensorgram,” which is a plot of response (measured in “resonance units” or “RU”) as a function of time. An increase of 1000 RU corresponds to an increase of mass on the sensor surface of approximately 1 ng/mM².

A detailed discussion of the technical aspects of the BIACORE instrument and the phenomenon of SPR may be found in U.S. Pat. No. 5,313,264. More detailed information on matrix coatings for biosensor sensing surfaces is given in, for example, U.S. Pat. Nos. 5,242,828 and 5,436,161. In addition, a detailed discussion of the technical aspects of the biosensor chips used in connection with the BIACORE instrument may be found in U.S. Pat. No. 5,492,840. The full disclosures of the above-mentioned U.S. patents are incorporated by reference herein.

In the following Examples, various aspects of the present invention are disclosed more specifically for purposes of illustration and not limitation.

### EXAMPLE 1

### Activation of a Sensor Surface (Entire Surface)

A sensor chip CM5 (a biosensor chip having a gold surface with a covalently linked carboxymethyl-modified dextran polymer hydrogel; Biacore AB, Uppsala, Sweden) was rinsed with deionized water and dried by a stream of nitrogen. 50 μl of a fresh solution of 0.2 M N-ethyl-N′-(dimethylaminopropyl)carbodiimide (EDC) and 0.05 M N-hydroxysuccinimide (NHS) were added to the surface. The solution covered the entire surface as a thin film of liquid. Immediately after adding the EDC/NHS solution, the sensor chip was put in a sealed box with high humidity to prevent evaporation from the surface. After 20 minutes, the sensor chip was taken out of the sealed box, rinsed with deionized water and dried by a stream of nitrogen. The dried sensor chip was stored at dry conditions for up to 8 hours before immobilization.

The contact angle of the chip surface was measured before and after NHS/EDC activation with a contact angle measuring device (FTA200, First Ten Angstrom, U.S.A.). Before activation, the contact angle of the sensor chip CM5 surface was 10°, and after activation 24°.

### EXAMPLE 2

### Activation of a Sensor Surface (Predefined Regions)

A sensor chip CM5 (Biacore AB) was rinsed with ionized water, dried by a stream of nitrogen and mounted on a holder. 100×100 pl of a fresh solution of 0.2 M EDC and 0.05 M NHS were added, using an ink-jet device (AutoDrop-Micropipette AD-K-501, Microdrop, Germany) to the surface. The EDC/NHS solution was located as a 10×10 matrix, with a pitch of 200 μm. The diameter of the spots was approximately 100 μm. Immediately after adding the EDC/NHS solution, the sensor chip was put in a sealed box with high humidity to prevent evaporation from the surface. After 20 minutes, the sensor chip was taken out of the sealed box, rinsed with deionized water and dried by a stream of nitrogen. The dried sensor chip was stored under dry conditions for up to 4 hours before immobilization was performed.

### EXAMPLE 3

### Immobilization of Antibodies to an Activated Surface

A sensor chip CM5 (Biacore AB) that had been activated according to Example 1 or Example 2 above was mounted to a holder. 100 pl of a solution of 10 mg/ml solution of anti-myoglobin antibody in 10 mM sodium acetate, pH 5.0, were added to the surface using the ink-jet device in Example 2. The diameter of the spots was approximately 100 μm. Immediately after adding the antibody solution, the sensor chip was put in a sealed box with high humidity to prevent evaporation from the surface. After 24 hours, the sensor chip was taken out of the sealed box, rinsed with ionized water and dried by a stream of nitrogen. When comparing the response for the immobilized spots with the response for non-immobilized regions on the sensor chip, a difference of about 5000 RU was obtained, corresponding to about 5 ng/mm² of immobilized antibody.

### EXAMPLE 4

### Activation of a Sensor Surface with Different Activating Agents

A sensor chip CM5 (Biacore AB) was activated as described in Example 1 but using other activating agents than NHS/EDC, and the contact angles of the prepared surfaces were measured with the measuring device used in Example 1. The contact angle of the sensor chip CM5 surface before activation was 10°, and the contact angles obtained after activation with the different activating agents are given in Table 1 below.

TABLE 1

| Activating agent | Contact angle |
|---|---|
| 3-Hydroxy-3,4-dihydrobenzotriazole | 38° |
| 1-Hydroxy-7-azabenzotriazole | 43° |
| 1-Hydroxybenzotriazole | 36° |
| Sodium phenol-4-sulfonate | 19° |
| Sodium 2-nitrophenol-4-sulfonate | 45° |

It is to be understood that the invention is not limited to the particular embodiments of the invention described above, but the scope of the invention will be established by the appended claims.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1.-30. (canceled)

31. A method of preparing an array of at least one binding agent on a solid support surface, which method comprises the steps of:
(1) providing a solid support having a surface area bearing a functional group, which surface area is hydrophilic having a contact angle with water of less than about 25 degrees,
(2) reacting the functional group on the solid support surface area with an activating agent to form a reactive group capable of coupling the binding agent or agents, wherein the activating agent is selected to form a reactive group which is sufficiently less polar than the functional group to make the activated solid support hydrophobic having a contact angle with water of at least about 40 degrees,
(3) selectively dispensing at each of a plurality of predefined discrete regions on the activated solid support surface area a predetermined volume of an aqueous liquid medium containing binding agent to couple the binding agent to the solid support surface area via the reactive group, the hydrophobic state of the activated surface area substantially preventing extension and spreading of the aqueous liquid medium when applied thereto, and
(4) deactivating unreacted reactive groups on the solid support surface area to more polar groups to make the solid support surface area hydrophilic having a contact angle with water of less than about 25 degrees.

32. The method according to claim 31, wherein the functional group is sufficiently polar to make the solid support surface area in step (1) is hydrophilic.

33. The method according to claim 31, wherein in step (3) the binding agent is covalently coupled to the reactive group.

34. The method according to claim 31, wherein the solid support surface area before activation of the functional group has a contact angle of less than about 20 degrees.

35. The method according to claim 31, wherein the activated surface area has a contact angle of higher than about 50 degrees.

36. The method according to claim 31, wherein the functional group is selected from carboxy, hydroxy, formyl, amino and mercapto groups.

37. The method according to claim 31, wherein the activating agent is capable of activating the functional group to a reactive group selected from hydroxysuccinimide ester, nitrophenyl ester, dinitrophenyl ester, tosylate, mesylate, triflate and disulfide groups.

38. The method according to claim 31, wherein the predefined discrete regions are spots having a diameter in the range of from about 10 to about 1000 μm.

39. The method according to claim 31, wherein the solid support comprises a gold film.

40. The method according to claim 31, wherein the solid support surface comprises extending polymer chains which bear a functional group.

41. The method according to claim 40, wherein the polymer forms a hydrogel.

42. The method according to claim 31, wherein the aqueous liquid medium containing a binding agent is deposited onto the predefined regions by a deposit device.

43. The method according to claim 42, wherein the deposit device is selected from devices based on ink-jet printing, micro-contact printing, capillary stamping, and inertia-driven ejection of microdrops.

44. The method according to claim 31, wherein the solid support surface comprises a sensor surface.

45. The method according to claim 31, wherein the sensor surface is a sensing surface of a biosensor.

46. The method according to claim 45, wherein the biosensor is based on evanescent wave sensing.

47. The method according to claim 45, wherein the biosensor is based on surface plasmon resonance.
